(19) United States
(12) Patent Application Publication
(10) Pub. No.: US 2024/0325189 A1
(43) Pub. Date: Oct. 3, 2024

(54) DRAIN POUCHING FLANGE AND POUCH SYSTEM

(71) Applicant: Sanguine Technology, LLC, Dunmore, PA (US)

(72) Inventors: Rosemary Granet, Scranton, PA (US); Paul Jason Granet, Scranton, PA (US); Gregory M. Murphy, Davidsonville, MD (US)

(21) Appl. No.: 18/738,027

(22) Filed: Jun. 9, 2024

Related U.S. Application Data
(63) Continuation-in-part of application No. 18/401,517, filed on Dec. 31, 2023.
Application No. 18/401,517 is a continuation of application No. 17/367,379, filed on Jul. 4, 2021, now Pat. No. 11,857,454.
Application No. 17/367,379 is a continuation-in-part of application No. 16/849,909, filed on Apr. 15, 2020, now Pat. No. 11,129,744.
(60) Provisional application No. 62/871,670, filed on Jul. 8, 2019.

(51) Int. Cl. A61F 5/448

(52) U.S. Cl. CPC A61F 5/448

(57) ABSTRACT

The present invention is directed to a device for protecting a user's or patient's skin and collecting effluent from a bodily drain tube including: a substrate, comprising a generally planar shape with two surfaces, a first surface of the substrate comprising adhesive for attachment to a user or patient; and a flange, disposed on and attached to the substrate, the flange being disposed on a second surface of the substrate.

### RELATED APPLICATIONS

The present application is a continuation-in-part of U.S. application Ser. No. 18/401,517, filed on 31 Dec. 2023, entitled “Ostomy Faceplates Configured for Use in Close Proximity With Creases, Rolls, or Folds,” which is a continuation of U.S. patent application Ser. No. 17/367,379, now U.S. Pat. No. 11,857,454, filed on 4 Jul. 2021, which is a continuation-in-part of U.S. patent application Ser. No. 16/849,909, now U.S. Pat. No. 11,129,744, filed on 28 Sep. 2021, which claims priority to U.S. Provisional Patent Application No. 62/871,670, filed on 8 Jul. 2019. Each of these references is incorporated by reference herein in their entirety.

The present application is also a continuation-in-part of Ser. No. 18/401,519, filed on 31 Dec. 2023, entitled “Improved Ostomy Appliance,” which is a continuation of U.S. patent application Ser. No. 17/480,768, now U.S. Pat. No. 11,857,453, filed on 21 Sep. 2021, which is a continuation of U.S. patent application Ser. No. 16/849,909, now U.S. Pat. No. 11,129,744, filed on 28 Sep. 2021, which claims priority to U.S. Provisional Patent Application No. 62/871,670, filed on 8 Jul. 2019. Each of these references is incorporated by reference herein in their entirety.

### FIELD OF THE INVENTION

The present invention is directed to a flange and pouch system for bodily drain tubes, wounds, ostomies, and/or other bodily features. More specifically, the present invention is directed to a flange with a break or opening in the perimeter and a pouch that attaches around the break or opening to hold the flange in a closed position. The pouch may comprise an opening for insertion of tubing. The opening may comprise an additional component, such as a silicone portion which may further comprise an aperture, opening, cut(s) or slit(s), etc. to receive tubing or other components therethrough.

### BACKGROUND

During various medical procedures, tubes, catheters, drains, etc. may be inserted into the body for a number of purposes. For example, a gastrostomy tube (or “g-tube”) may be utilized to provide nutrition directly to a patient's stomach. A nephrostomy patient may have a catheter connected to an upper portion of their urinary tract. Drain tubes may be utilized around certain wounds or incisions. In these cases, there may be leakage from around the tube, catheter, drain, etc. Such leakage may irritate the proximate skin and cause additional inconveniences, such as wetting clothing, causing undesirable odors, etc. Accordingly, it is desirable to capture such leakage from around such devices.

### SUMMARY OF THE INVENTION

In accordance with some embodiments of the present invention, a device for protecting a user's or patient's skin and collecting effluent from a bodily drain tube including: a substrate, comprising a generally planar shape with two surfaces, a first surface of the substrate comprising adhesive for attachment to a user or patient; and a flange, disposed on and attached to the substrate, the flange being disposed on a second surface of the substrate may be used.

Some aspects of the present invention may include a barrier with a flange with an open or “broken” perimeter and a pouch with a channel or mating flange. The pouch may further comprise an opening for insertion of a tube or catheter.

These and other aspects will become apparent from the following description of the invention taken in conjunction with the following drawings, although variations and modifications may be effected without departing from the spirit and scope of the novel concepts of the invention.

### DESCRIPTION OF THE FIGURES

The present invention can be more fully understood by reading the following detailed description together with the accompanying drawings, in which like reference indicators are used to designate like elements. The accompanying figures depict certain illustrative embodiments and may aid in understanding the following detailed description. Before any embodiment of the invention is explained in detail, it is to be understood that the invention is not limited in its application to the details of construction and the arrangements of components set forth in the following description or illustrated in the drawings. The embodiments depicted are to be understood as exemplary and in no way limiting of the overall scope of the invention. Also, it is to be understood that the phraseology and terminology used herein is for the purpose of description and should not be regarded as limiting. The detailed description will make reference to the following figures, in which:

FIG. 1 illustrates an exemplary barrier in accordance with some embodiments of the present invention.

FIG. 2 illustrates an exemplary barrier in accordance with some embodiments of the present invention.

FIG. 3 illustrates an exemplary break in a barrier flange, in accordance with some embodiments of the present invention.

FIG. 4 illustrates an exemplary break in a barrier flange, in accordance with some embodiments of the present invention.

FIG. 5 illustrates an exemplary break in a barrier flange, in accordance with some embodiments of the present invention.

FIG. 6 illustrates an exemplary interaction between a flange on a barrier and a channel on a pouch, in accordance with some embodiments of the present invention.

FIG. 7 illustrates an exemplary interaction between a flange on a barrier and a channel on a pouch, in accordance with some embodiments of the present invention.

FIG. 8 illustrates an exemplary interaction between a flange on a barrier and a channel on a pouch, in accordance with some embodiments of the present invention.

FIG. 9 illustrates an exemplary pouch that may be used in accordance with some embodiments of the present invention.

FIG. 10 illustrates an exemplary pouch that may be used in accordance with some embodiments of the present invention.

FIG. 11 illustrates exemplary orifices or openings for a pouch and/or additional element, in accordance with some embodiments of the present invention.

FIG. 12 illustrates an exemplary system, comprising a barrier and pouch installed around tubing or catheter, in accordance with some embodiments of the present invention.

FIGS. 13A-13D illustrate various forms or shapes of barriers and flanges, in accordance with some embodiments of the present invention.

FIG. 14 illustrates an exemplary open pouch that can be opened to be installed around a preexisting tubing or catheter, in accordance with some embodiments of the present invention.

### DETAILED DESCRIPTION

Before any embodiment of the invention is explained in detail, it is to be understood that the present invention is not limited in its application to the details of construction and the arrangements of components set forth in the following description or illustrated in the drawings. The present invention is capable of other embodiments and of being practiced or being carried out in various ways. Also, it is to be understood that the phraseology and terminology used herein is for the purpose of description and should not be regarded as limiting.

The matters exemplified in this description are provided to assist in a comprehensive understanding of various exemplary embodiments disclosed with reference to the accompanying figures. Accordingly, those of ordinary skill in the art will recognize that various changes and modifications of the exemplary embodiments described herein can be made without departing from the spirit and scope of the claimed invention. Descriptions of well-known functions and constructions are omitted for clarity and conciseness. Moreover, as used herein, the singular may be interpreted in the plural, and alternately, any term in the plural may be interpreted to be in the singular.

As discussed in greater detail below, the flange may have a single break, or may be configured to have one or two overlapping elements. The overlapping portions of the flange may be configured to mate together, or may simply be pressed together by a mating flange.

In order to install a device around existing tubes, catheters, drains, etc., it may be helpful to have a flange that is broken, such that the flange can be separated at the break and slipped around the tube, catheter, drain, etc. A mating portion of the flange on a pouch may then hold the broken flange in a closed position to prevent or reduce leakage.

In addition, it is contemplated that pouches or bags may be specifically designed and provided to adapt to the use around existing tubing, catheters, etc. For example, pouches may be provided with an aperture or opening therein configured to receive tubing therethrough. To prevent leakage, as well as provide additional support for the tubing, catheters, etc., additional components may be provided on the pouch itself. For example, it is contemplated that an additional element may be provided with an opening, cut, or slit therethrough, configured to receive tubing. The opening, cut, or slit may be of any configuration (a round opening, a single slit, two intersecting slits, several cuts or slits causing a star pattern, etc.), and in accordance with some embodiments of the present invention, may be sized slightly smaller than a diameter of tubing to be used therewith, thereby providing a friction fit of the tubing.

For example, a silicone pad may be attached to an exterior of a pouch. An opening may be disposed in the pouch aligned with the silicone pad. The silicone pad may have an opening sized to receive the tubing therethrough. Other elements or materials may be used, and may be disposed on the outside of the pouch or the inside of the pouch. For certain applications, the additional element may provide additional features, such as odor control. For example, an activated carbon element may be used to support the tubing and provide odor control.

In an effort to prevent or reduce leakage, it is also contemplated that the additional element—such as but not limited the silicone pad or carbon element as discussed above—may be configured to repel liquid, for example by being treated with a hydrophobic coating or having natural hydrophobic properties.

The flange itself may be in any shape or configuration. It is in contemplated that a shape may be selected to reduce the amount of area it covers. For example, a triangular or oval shape may reduce the amount of area covered by the barrier or flange, while providing adequate skin protection and effluent collection. Shapes may also be rectangular, capsule shaped (for example, two parallel sides connected by arcs or rounded sides), D-shaped, etc.

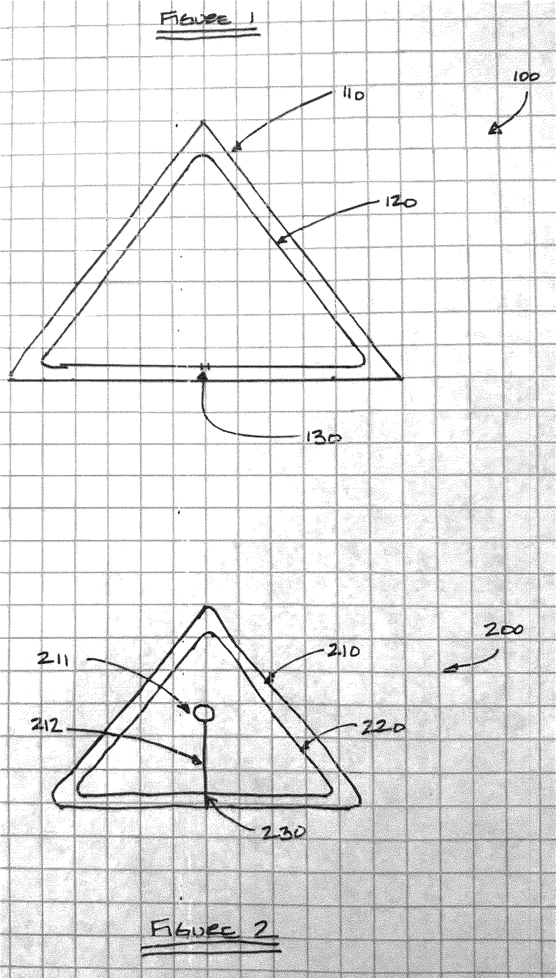

With reference to FIG. 1, a barrier 100 will now be described. Barrier 100 may comprise a barrier material 110, such as but not limited to a hydrocolloid or hydrogel component, foam, film, etc. Such barrier material may have one side coated in an adhesive, in order to adhere to a patient's skin. The side of the barrier material opposite to the adhesive may have a flange 120, in order to permit the attachment of a pouch or bag to collect any effluent. In accordance with some embodiments of the present invention, the flange may be an extrusion, which may include additional elements, latches, clips, etc., as discussed more thoroughly below. In some embodiments, the flange may have a break 130.

With reference to FIG. 2, it is contemplated that a barrier 200 may be provided with an opening 211 to surround tubes, catheters, etc., as well as a cut 212 in the barrier material in-line with the break or opening 230 in a flange 220. In this manner, the barrier may be separated about the break, and inserted around a tube, catheter, etc., which may be disposed in the opening 211. Using the break in the flange 230 and the slit 212 in the barrier material 210, the barrier 200 may be placed around a pre-existing tube, catheter etc.

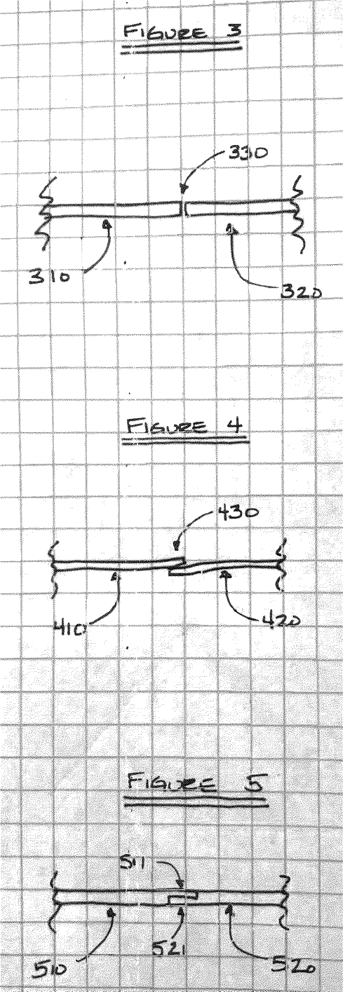

FIGS. 3-5 illustrate examples of different breaks in the flange, as contemplated by the present invention. FIG. 3 shows a flange with two portions 310, 320, separated by a break 330. FIG. 3 shows the two portions 310, 320 butted against each other.

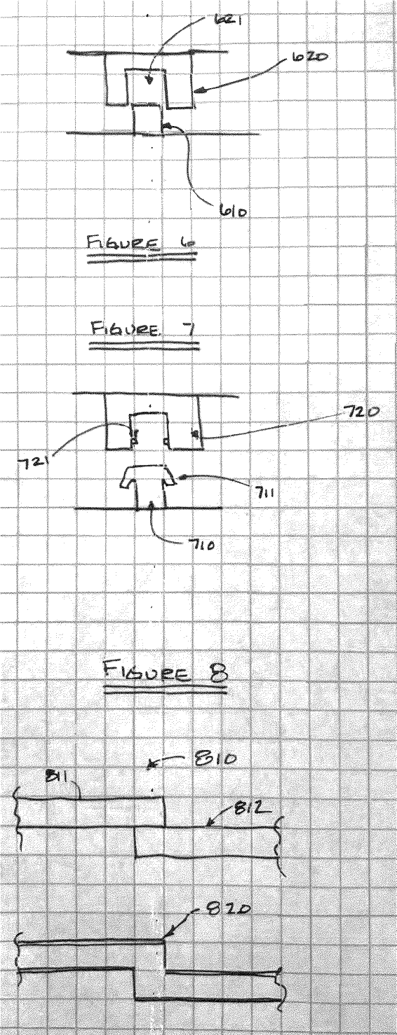

FIG. 4 illustrates an overlapping joint. Portions 410, 420 overlap each other at 430. FIG. 5 illustrates two portions 510, 520 with an overlapping lap joint, formed from portions of the flange with approximately halved thickness at 511, 521.

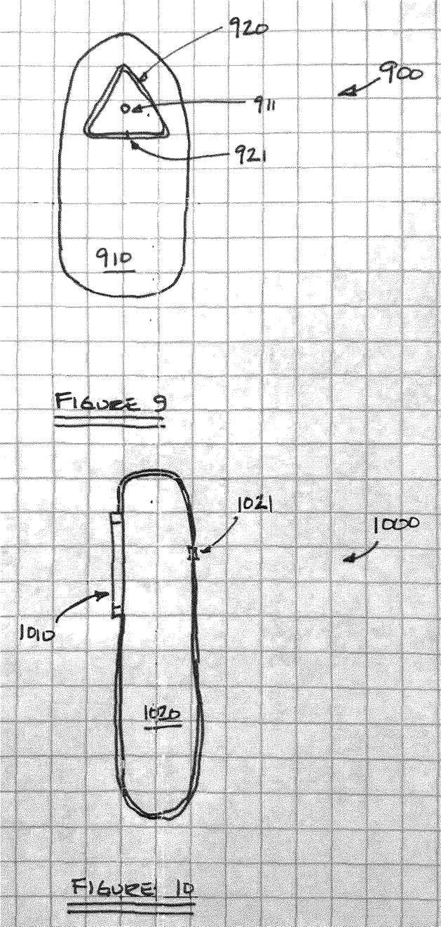

FIGS. 6-7 illustrate an exemplary cross-section of a flange portion on a barrier with the receiving flange on a pouch. With reference to FIG. 6, extrusion 610 shows an exemplary cross section of a flange that may be disposed on a barrier. Receiving extrusion 620 may comprise a channel 621 that may be disposed on a pouch, configured to receive the extrusion on the barrier. With reference to FIG. 7, additional elements are shown. Again, a flange extrusion 710 may extend from a barrier, and may include spring elements 711 that may be compressed upon insertion into channel 720. Spring elements may engage protrusions 721 such that the flange is locked into the channel until forcibly removed by a user.

FIG. 8 illustrates a top view of an overlapping flange 810 (similar to that shown in FIG. 4), showing the overlap of flange portions 811, 812. The channel to receive the overlapping flanges is shown at 820, and may match the outline of the overlapping flange elements.

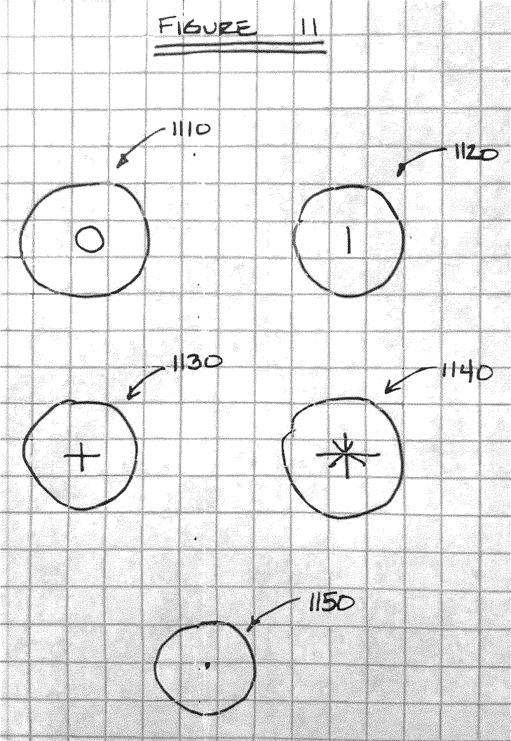

With reference to FIG. 9, an exemplary pouch 900 is illustrated. Pouch 900 may comprise a bag material 910 with a channel or mating flange 920 disposed thereon. The channel or mating flange 920 may include a portion 921 configured to receive the break or overlap in the flange on the barrier. Pouch 910 may also comprise an opening 911 in line with the flange so that the tubing or catheter can extend through the pouch.

Looking to FIG. 10, a side view of an exemplary pouch 1000 will now be discussed. Exemplary pouch 1000 may comprise a channel or mating flange 1010 on one side, and an opening (for example, a hole, orifice, slit(s), cut(s), etc.) 1021 on the other side. Pouch may have an open interior 1020 for receiving effluent.

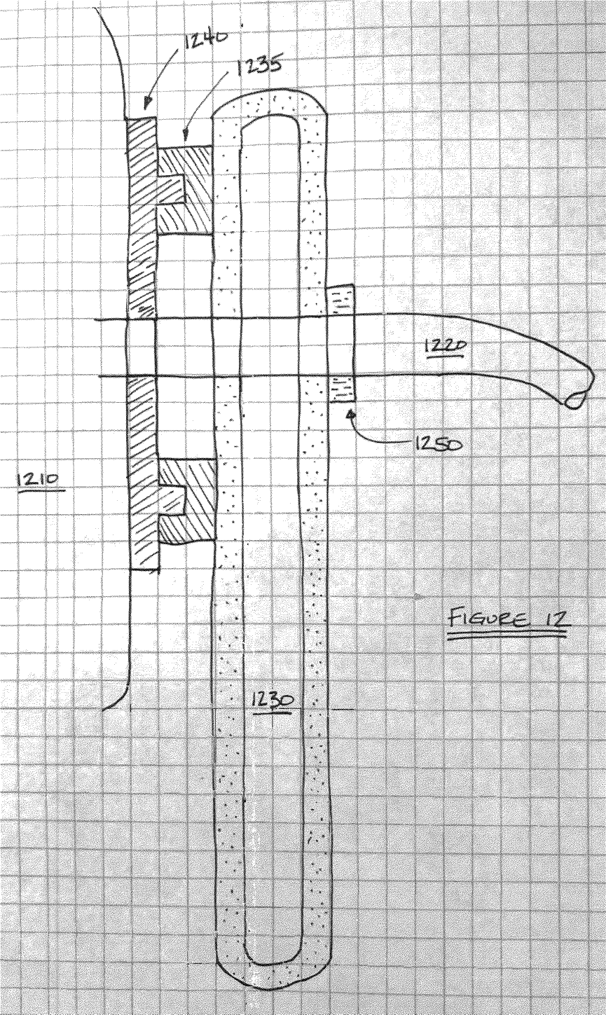

As noted above, there may be an opening in the side of the pouch for the insertion of a tube, catheter, etc. therethrough. In order to support the tube, catheter, etc., as well as providing additional sealing, an additional element may be utilized. Regardless of whether the opening is only in the pouch itself or also in an additional element, the opening or orifice may take any shape or size. For example, with reference to FIG. 11, the opening or orifice may comprise a hole 1110 (which, for example, may be sized approximately the same diameter as the expected tubing, catheter, etc., (or slightly less). The opening may be a slit 1120, two intersecting slits 1130, or even multiple slits causing a star patter 1140. Depending on the material, the opening may also be a pinhole opening or puncture 1150. For example, in the case of a silicone element, the elastic nature of silicone may permit the tubing or catheter to be inserted into the puncture 1150.

FIG. 12 illustrates an cross sectional view of an exemplary barrier and pouch system. A barrier 1240 may be attached to a patient 1210, typically through the use of an adhesive. A pouch 1230 may attach thereto via a channel or mating flange 1235. Tube or catheter 1220 may extend from the body 1210 through the barrier 1240 and through the pouch 1230. The pouch 1230 may include an additional element 1250 that may be utilized to support and/or seal the tube or catheter 1220.

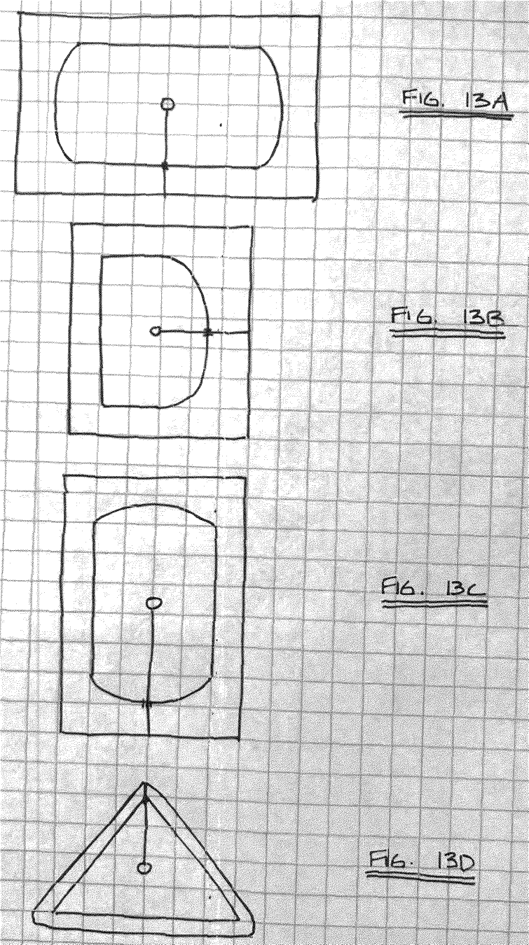

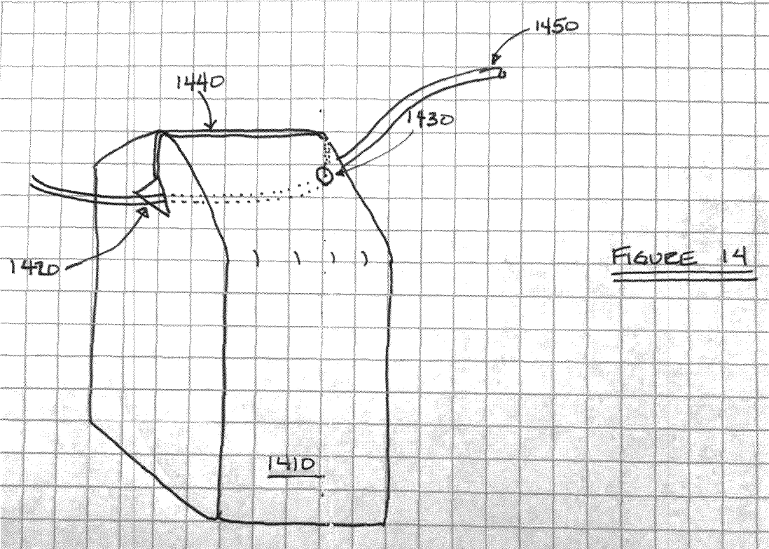

With reference to FIGS. 13A-13D, various configurations of a barrier with flange are shown. The flange may take any shape, and the barrier material (such as but not limited to a hydrocolloid material) may be in any shape, and may be cut before provision to patient or by a patient or healthcare provider.

It is further contemplated that in some circumstances, it may be desirable to install a pouch to collect effluent around an existing drain tube or catheter where there is no accessible distal end to the tube or catheter to insert through an orifice or opening in the pouch. In such circumstances, the pouch may be installed around the tube or catheter. With reference to FIG. 14, an exemplary pouch with an open portion is illustrated. Pouch 1410 may comprise a channel or mating flange 1420 to attach to a barrier, as discussed above. The channel or mating flange 1420 may include a break in its perimeter, which may be aligned with a slit or cut opening 1440 in the pouch 1410. The slit or cut opening may run the top of the pouch 1410. In some embodiments, a support element 1430, such as discussed above, may also be used. The support element may include a slit running from a central position to an edge, aligned with the slit or cut opening 1440 such that a tube or catheter 1450 may be placed through the slit or cut, through the break in the perimeter of the channel or mating flange, and into the slit on the support element. The slit or cut 1440 in the pouch 1410 may then be sealed by various mechanisms. For example, each side of the slit or cut may have zip fastener (interlocking plastic strips, such as zip top bags, strips of adhesive, hook-and-loop fasteners, clamps (such as used in drainable ostomy pouches), clips, etc.

In this manner, a pouch may be installed over an existing tube or catheter when the distal end is not accessible.

It will be understood that the specific embodiments of the present invention shown and described herein are exemplary only. Numerous variations, changes, substitutions and equivalents will now occur to those skilled in the art without departing from the spirit and scope of the invention. Accordingly, it is intended that all subject matter described herein and shown in the accompanying drawings be regarded as illustrative only, and not in a limiting sense.

What is claimed:

1. A device for protecting a user's or patient's skin and collecting effluent from a bodily drain tube comprising:
a substrate, comprising a generally planar shape with two surfaces, a first surface of the substrate comprising adhesive for attachment to a user or patient;
a flange, disposed on and attached to the substrate, the flange being disposed on a second surface of the substrate.

2. The device of claim 1, wherein the substrate comprises a impermeable, substantially impermeable, or vapor permeable material.

3. The device of claim 1, further comprising an aperture in the substrate, disposed within the flange.

4. The device of claim 1, wherein the flange comprises a non-circular shape for the mechanical attachment of a pouch or bag.

5. The device of claim 1, wherein the flange comprises at least one substantially straight edge, the flange for the mechanical attachment of a pouch or bag.

6. The device of claim 1, wherein the flange is substantially “D”-shaped.

7. The device of claim 1, wherein the flange is substantially triangular shaped.

8. The device of claim 1, wherein the substrate comprises a hydrocolloid material.

9. The device of claim 1, wherein the flange is broken, to permit items to be inserted into the flange through the break.

10. The device of claim 3, wherein the substrate comprises a slit or cut aligned with the aperture.

11. The device of claim 10, wherein the flange comprises a break to permit items to be inserted into the flange through the break, and wherein the break in the flange aligns with the slit or cut in the substrate.
